(19) United States
(12) Patent Application Publication
(10) Pub. No.: US 2009/0137939 A1
(43) Pub. Date: May 28, 2009

(54) DEVICE FOR TREATING BLOOD IN AN EXTRACORPOREAL CIRCUIT

(75) Inventors: Edgardo Costa Maianti, Mirandola (IT); Nicola Ghelli, San Pietro in Casale (IT); Ivo Panzani, Mirandola (IT)

Correspondence Address: POPOVICH, WILES & O'CONNELL, PA;650 THIRD AVENUE SOUTH, SUITE 600, MINNEAPOLIS, MN, 55402, US

(73) Assignee: SORIN GROUP ITALIA S.R.L., Mirandola (IT)

(21) Appl. No.: 12/364,985

(22) Filed: Feb. 3, 2009

Related U.S. Application Data
(63) Continuation of application No. 10/614,722, filed on Jul. 7, 2003.

(30) Foreign Application Priority Data
Jul. 15, 2002 (IT) MI2002A001552

(51) Int. Cl. A61M 1/36

(52) U.S. Cl. 604/6.09

(57) ABSTRACT

A device for treating blood in an extracorporeal circuit comprising a venous blood reservoir having an inlet and an outlet, a heat exchanger having an inlet and an outlet, a pump having an inlet and an outlet, an oxygenation apparatus having an inlet and an outlet, and an arterial blood filter having an inlet and an outlet, wherein the venous blood reservoir, heat exchanger, pump, oxygenation apparatus, and arterial blood filter are integrated into a single monolithic assembly.

This application is a continuation of U.S. patent application Ser. No. 10/614,722, filed Jul. 7, 2003, the contents of which are hereby incorporated by reference herein.

### FIELD OF THE INVENTION

The present invention relates to a device for treating blood in an extracorporeal circuit.

### BACKGROUND OF THE INVENTION

It is known that during certain surgical procedures it is necessary to establish an extracorporeal circulation of the blood of the patient in a circuit that comprises devices suitable to ensure correct treatment of blood. Such devices comprise at least one reservoir for containing the blood, termed venous blood, that leaves the patient, a pump for conveying the blood in the circuit, a heat exchanger in which the blood encounters a heat exchange fluid that ensures its correct temperature, an oxygenation apparatus meant to transfer oxygen to the blood, and finally a filter that is interposed on the line, known as arterial blood line, that returns the blood to the patient, with the purpose of retaining any air bubbles that are present in the blood. The described extracorporeal circuit is completed by the presence of a container for the blood collected by salvage from the operating field, known as a cardiotomy reservoir, which is connected to the venous blood reservoir.

Originally, extracorporeal circuits comprised the described devices as independent elements that were interconnected by virtue of connecting lines. In order to improve the functional parameters of the circuit, particularly minimizing hemodilution, hemolysis and risk of embolisms, and in order to improve oxygen transport and streamline the distribution of the components around the operating field, partial integrations of the devices have been made in the prior art.

### SUMMARY OF THE INVENTION

The aim of the present invention is now to provide a device for treating blood in an extracorporeal circuit that allows minimizing the filling volume, i.e., the volume of the blood that is present outside the body of the patient, the surface in contact with the blood, and the overall dimensions, so as to ensure an optimum treatment of the blood in the circuit and convenient management on the part of operators. This aim can be achieved by a device for treating blood in an extracorporeal circuit, according to the invention, characterized in that it comprises, integrated in a single monolithic assembly, a venous blood reservoir that is connected to a line for conveying the blood that arrives from the patient, a heat exchanger, a pump, an oxygenation apparatus and an arterial blood filter connected to a line for returning the blood to the patient.

The invention provides a device for treating blood in an extracorporeal circuit comprising a venous blood reservoir having an inlet and an outlet, a heat exchanger having an inlet and an outlet, a pump having an inlet and an outlet, an oxygenation apparatus having an inlet and an outlet, and an arterial blood filter having an inlet and an outlet, wherein the venous blood reservoir, heat exchanger, pump, oxygenation apparatus, and arterial blood filter are integrated into a single monolithic assembly.

Additional features and advantages of the invention are set forth in the description which follows and in part will be apparent from the description. The objectives and other advantages of the invention will be realized and attained by the device for treating blood in an extracorporeal circuit particularly pointed out in the written description and claims.

It is to be understood that both the foregoing general description and the following detailed description are exemplary and explanatory and are intended to provide further explanation of the invention as claimed.

### BRIEF DESCRIPTION OF THE DRAWING

Further characteristics and advantages will become apparent from the description of two preferred but not exclusive embodiments of the invention which are illustrated by way of non-limitative example in the accompanying drawings.

FIG. 1 is a diagram of a prior art extracorporeal circuit.

FIGS. 2 and 4 are diagrams of different embodiments of an extracorporeal circuit that comprises the device according to the invention.

FIGS. 3 and 5 are cross-sectional views of the devices illustrated in FIGS. 2 and 4, respectively.

### BRIEF DESCRIPTION OF THE PREFERRED EMBODIMENTS

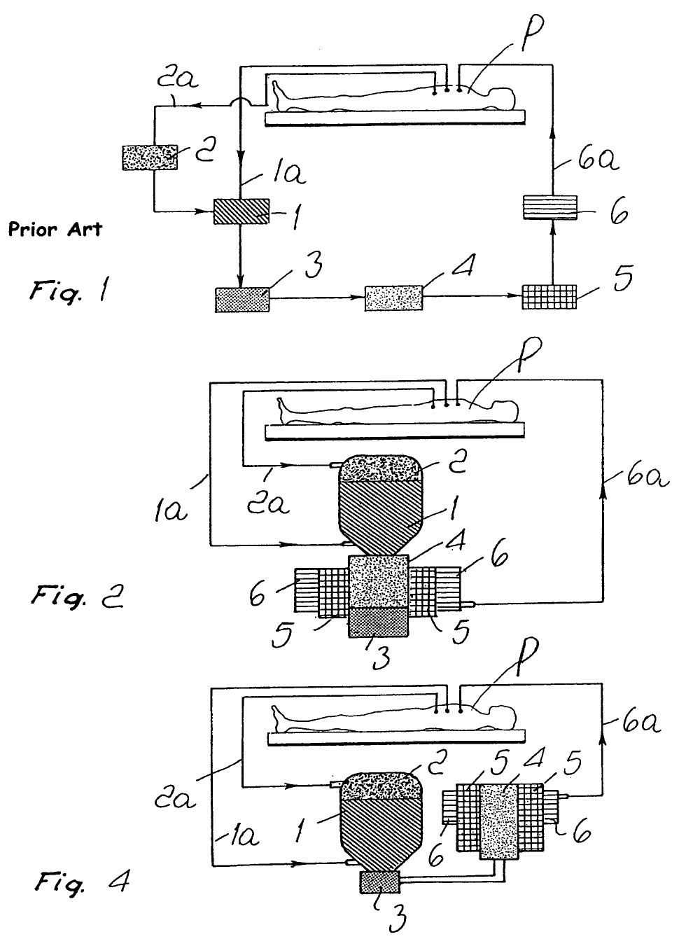

With reference to FIG. 1, the reference numeral 1a designates the venous blood line that conveys the blood from the patient P to the venous blood reservoir 1, which also receives blood from the cardiotomy reservoir 2 for collection from the operating field (i.e., from patient P) by means of the line 2a. The reference numeral 3 designates the pump, reference numeral 4 designates the heat exchanger, and reference numeral 5 designates the oxygenation apparatus, from which arterial blood line 6a leads out, with arterial blood filter 6 interposed, returning the blood to the patient P.

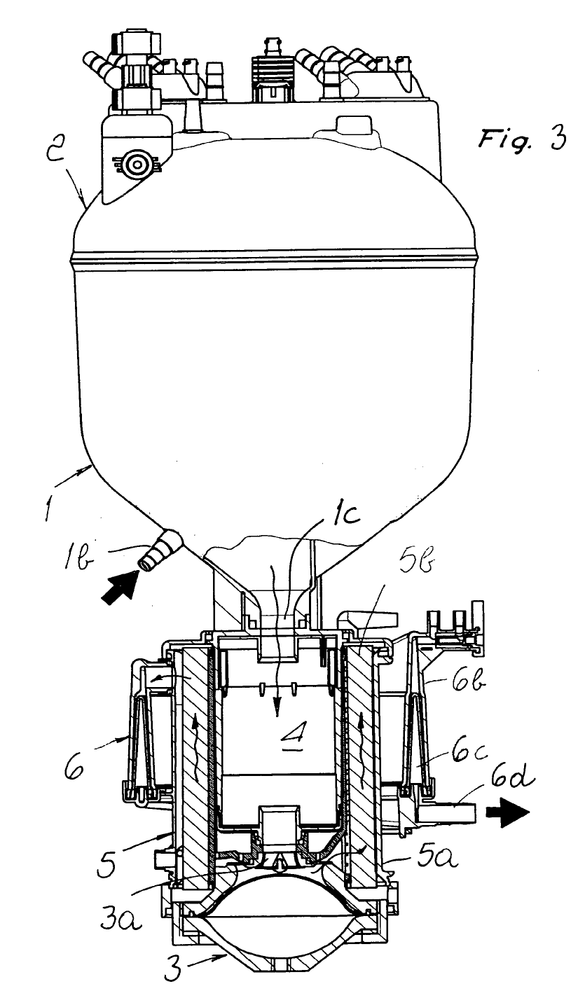

As mentioned above, originally extracorporeal circuits comprised all the devices as independent elements connected by connecting lines. According to this invention, the described devices are integrated into a single device and are designated, in the subsequent figures, by the same reference numerals adopted in FIG. 1. Furthermore, in FIGS. 2 and 4, the individual devices are shown with the same graphical identification as shown in FIG. 1.

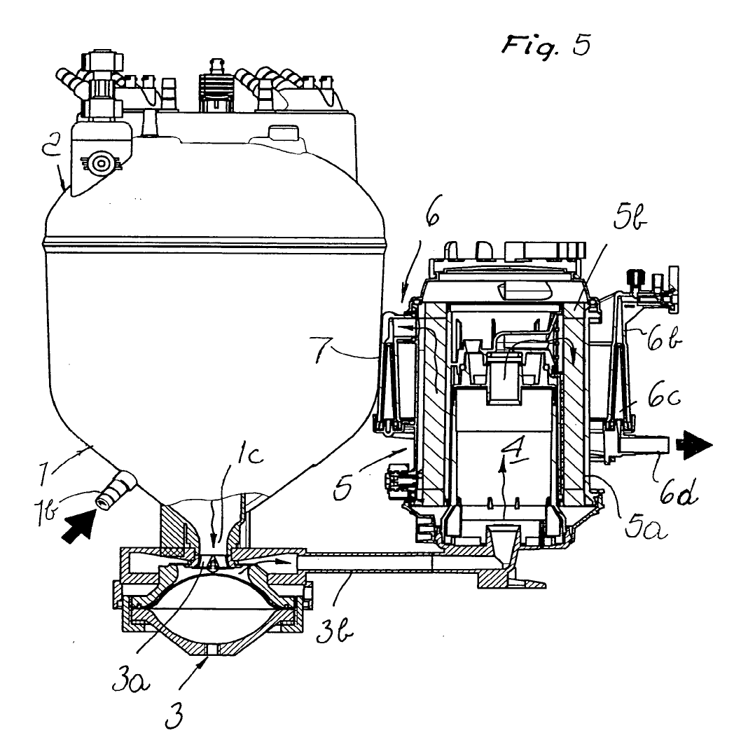

The first embodiment of the invention is described with reference to FIGS. 2 and 3. In this embodiment, oxygenation apparatus 5 comprises hollow cylindrical structure 5a for containing blood oxygenation structures 5b such as hollow fibers and is suitable to contain the heat exchanger 4. Structure 5a supports, at its upper face, venous blood reservoir 1, which is connected to the patient P by means of venous blood line 1a that reaches connector 1b, and supports, at its lower face, pump 3, which is of the pulsating type. There is also annular structure 6b of arterial blood filter 6, which is monolithically connected to structure 5a and is suitable to contain filtration structure 6c for the arterial blood that returns to the patient P by means of arterial blood line 6a that leads out from connector 6d. Cardiotomy reservoir 2 is integrated in a position above the venous blood reservoir 1 and is connected to the operating field by the line 2a. The venous blood reservoir 1 lid comprises a passage port that is provided with an occlusion device.

In the described device, the blood follows the arrows shown in FIG. 3, and therefore, once it has entered venous blood reservoir 1 from connector 1b, it is introduced by means of output connector 1c of the reservoir into heat exchanger 4, which has a coaxial and directly facing inlet connector. At the outlet of heat exchanger 4, the blood enters the coaxial and directly facing intake duct 3a of pump 3, is sent by the pump to the inlet of oxygenation apparatus 5, and then exits the apparatus and enters arterial blood filter 6 so as to be able to return to the patient P by means of arterial blood line 6a connected to connector 6d.

The second embodiment of the invention is now described with reference to FIGS. 4 and 5. In this embodiment, intake duct 3a of pump 3 is connected directly to output connector 1c of venous blood reservoir 1, which receives the blood from venous blood line 1a connected to inlet connector 1b and supports cardiotomy reservoir 2, which is integrated therein in an upward region. Delivery duct 3b of pump 3 ends at the base of hollow cylindrical structure 5a of oxygenation apparatus 5, which contains blood oxygenation structures 5b such as hollow fibers for blood oxygenation, and is suitable to accommodate heat exchanger 4. Annular structure 6b of arterial blood filter 6 is provided at the peripheral region of structure 5a and is monolithically connected thereto. The structure 6b is suitable to contain filtration structure 6c for filtering the arterial blood that returns to the patient P by means of arterial blood line 6a connected to connector 6d. As shown in FIG. 5, annular structure 6b is monolithically connected to venous blood reservoir 1 at portion 7.

The arrows of FIG. 5 illustrate the path of the blood, which is introduced in pump 3 from venous blood reservoir 1 and is conveyed by delivery tube 3b of the pump so that it arrives at the base of structure 5a at the axis thereof, thus facing directly the inlet of heat exchanger 4. When the blood leaves exchanger 4, it enters oxygenation apparatus 5 and then passes into arterial blood filter 6, subsequently returning to the patient P by means of arterial blood line 6a connected to connector 6d.

The above description and accompanying drawings are provided for the purpose of describing embodiments of the invention and are not intended to limit the scope of the invention in any way. It will be apparent to those skilled in the art that various modifications and variations can be made in the device for treating blood in an extracorporeal circuit without departing from the spirit or scope of the invention. For example, the pulsating pump can be replaced with a centrifugal pump. Thus, it is intended that the present invention cover the modifications and variations of this invention provided they come within the scope of the appended claims and their equivalents.

What is claimed is:

1. A device for treating blood in an extracorporeal circuit comprising a venous blood reservoir having an inlet and an outlet, a heat exchanger having an inlet and an outlet, a pump having an inlet and an outlet, an oxygenation apparatus having an inlet and an outlet, and an arterial blood filter having an inlet and an outlet,
wherein the venous blood reservoir, heat exchanger, pump, oxygenation apparatus, and arterial blood filter are integrated into a single monolithic assembly,
wherein the outlet of the venous reservoir is connected to the inlet of the pump, the outlet of the pump is connected to the inlet of the heat exchanger, the outlet of the heat exchanger is connected to the inlet of the oxygenation apparatus, and the outlet of the oxygenation apparatus is connected to the inlet of the arterial filter,
wherein the inlet of the pump is connected directly to the outlet of the venous blood reservoir, the outlet of the pump ends at the base of a first hollow cylindrical structure for containing a blood oxygenation structure,
wherein the first hollow cylindrical structure is suitable to accommodate the heat exchanger and to support, at the peripheral region, a second hollow cylindrical structure that is suitable to contain a filtration structure for filtering the arterial blood, and
wherein the pump is placed below the venous reservoir and at a level lower than the oxygenation apparatus.

2. The device according to claim 1, further comprising a cardiotomy reservoir that is monolithically connected to the venous blood reservoir.

3. The device according to claim 1, wherein the outlet of the pump ends at the base of the first hollow cylindrical structure at the axis thereof and directly faces the inlet of the heat exchanger provided within the first hollow cylindrical structure.

4. The device according to claim 1, wherein the pump is a pulsating pump.

5. The device according to claim 1, wherein the pump is a centrifugal pump.
